(19) United States
(12) Patent Application Publication
(10) Pub. No.: US 2008/0071185 A1
(43) Pub. Date: Mar. 20, 2008

(54) Periodic breathing during activity

(75) Inventors: Kenneth C. Beck, St. Paul, MN (US); Kent Lee, Shoreview, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., ST Paul, MN (US)

(21) Appl. No.: 11/900,049

(22) Filed: Sep. 7, 2007

Related U.S. Application Data
(63) Continuation-in-part of application No. 11/463,076, filed on Aug. 8, 2006.

(51) Int. Cl. A61B 5/08

(52) U.S. Cl. 600/529

(57) ABSTRACT

An implantable respiration monitor can detect disordered breathing events that can be categorized, such as according to one or more of sleep, exercise, and resting awake states. The categorized frequency of such events can be compared to independently specifiable thresholds, such as to trigger an alert or responsive therapy, or to display one or more trends. The information can be combined with detection of one or more other congestive heart failure (CHF) symptoms to generate a CHF status indicator or to trigger an alarm or responsive. The alert can notify the patient or a caregiver, such as via remote monitoring. Respiration patterns from one or more of the activity states can be used to establish model of disordered breathing to which further respiration data can be compared for identifying periods of disordered breathing. Such identification can trigger an alert or response to therapy, or to display one or more trends.

### CROSS-REFERENCE TO RELATED APPLICATIONS

This patent application is a continuation-in-part of Pu et al. U.S. patent application Ser. No. 11/463,076, filed on Aug. 8, 2006, entitled “RESPIRATION MONITORING FOR HEART FAILURE USING IMPLANTABLE DEVICE,” and assigned to Cardiac Pacemakers, Inc., and the disclosure of the above referenced patent application is incorporated herein by reference in its entirety.

### TECHNICAL FIELD

This patent document pertains generally to disordered breathing and congestive heart failure and more particularly, but not by way of limitation, to categorizing, such as by using sleep or exercise states, respiration monitored using implantable device, such as for heart failure status monitoring.

### BACKGROUND

Sleep is generally beneficial and restorative to a person. Therefore, it exerts a great influence on a person's quality of life. The human sleep/wake cycle generally conforms to a circadian rhythm that is regulated by a biological clock. Regular periods of sleep enable the body and mind to rejuvenate and rebuild. The body may perform various tasks during sleep, such as organizing long term memory, integrating new information, and renewing tissue and other body structures.

Lack of sleep and/or decreased sleep quality may have a number of causal factors including, e.g., respiratory disturbances, nerve or muscle disorders, and emotional conditions, such as depression and anxiety. Chronic long-term sleep-related disorders such as chronic insomnia, sleep-disordered breathing, and sleep movement disorders may significantly affect a patient's sleep quality and quality of life.

Sleep apnea, for example, is a fairly common breathing disorder characterized by periods of interrupted breathing experienced during sleep. Sleep apnea is typically classified based on its etiology. One type of sleep apnea, denoted as obstructive sleep apnea, occurs when the patient's airway is obstructed by the collapse of soft tissue in the rear of the throat. Central sleep apnea is caused by a derangement of the central nervous system control of respiration. The patient ceases to breathe when control signals from the brain to the respiratory muscles are absent or interrupted. Mixed apnea is a combination of the central and obstructive sleep apnea types. Regardless of the type of apnea people experiencing an apnea event stop breathing for a period of time. The cessation of breathing may occur repeatedly during sleep, sometimes hundreds of times a night and occasionally for a minute or longer.

In addition to apnea, other types of disordered breathing have been identified, including, for example, hypopnea (shallow breathing), dyspnea (labored breathing), hyperpnea (deep breathing), and tachypnea (rapid breathing). Combinations of the disordered respiratory events described above have also been observed. For example, Cheyne-Stokes respiration (CSR, which is sometimes referred to as periodic breathing) is associated with rhythmic increases and decreases in tidal volume caused by alternating periods of hyperpnea followed by apnea or hypopnea. The breathing interruptions of CSR may be associated with central apnea, or may be obstructive in nature. CSR is frequently observed in patients with congestive heart failure (CHF) and is associated with an increased risk of accelerated CHF progression.

### Overview

An implantable respiration monitor can be used to detect disordered breathing or periodic breathing events that can be categorized, such as according to one or more of sleep, exercise, or resting awake states. The categorized frequency of such events can be compared to independently specifiable thresholds, such as to trigger an alert or responsive therapy, or to display one or more trends. The information can also be combined with detection of one or more other congestive heart failure (CHF) symptoms, such as to generate a CHF status indicator or to trigger an alarm or responsive therapy or to display one or more trends. The alert can notify the patient or a caregiver, such as via remote monitoring. The sleep state information can be further categorized according to central sleep apnea (CSA) or obstructive sleep apnea (OSA) events.

Example 1 includes a method comprising monitoring respiration of a subject, detecting physical activity of the subject, obtaining a respiration pattern of the subject during the activity, and analyzing how well the respiration pattern fits a model. The analyzing providing a goodness of fit indication, identifying a disordered breathing during activity indication from the goodness of fit indication, and providing the disordered breathing during activity indication to a user or automated process.

In Example 2, the method of Example 1 is optionally performed such that obtaining a respiration pattern comprises determining tidal volume of the subject.

In Example 3, the method of Example 1 is optionally performed such that obtaining a respiration pattern comprises determining respiration rate of the subject.

In Example 4, the method of Example 1 is optionally performed such that obtaining a respiration pattern comprises determining minute ventilation of the subject.

In Example 5, the method of Example 1 is optionally performed such that detecting physical activity comprises detecting a period of sustained physical activity exceeding an exertion or duration specified by a user.

In Example 6, the method of Examples 1 or 5 is optionally performed such that the specified exertion comprises activity exceeding at least 20 mGs.

In Example 7, the method of Examples 1 or 5 is optionally performed such that the specified duration comprises at least three minutes.

In Example 8, the method of Example 1 is optionally performed such that analyzing how well the respiration pattern fits the model comprises using a model of a respiration signal over time.

In Example 9, the method of Example 1 is optionally performed such that analyzing how well the respiration pattern fits the model comprises using a model of a respiration within the frequency domain.

In Example 10, the method of Example 1 is optionally performed such that providing the goodness of fit indication comprises applying one or more of a least squares analysis or a power spectrum analysis to obtain the goodness of fit indication.

In Example 11, the method of Example 1 is optionally includes reporting a respiration pattern magnitude, a respiration pattern cycle rate, or a respiration pattern cycle length in response to the goodness of fit indication meeting at least one criterion.

In Example 12, the method of Example 1 is optionally includes automatically delivering a response to the subject in response to the periodic breathing during activity indication.

In Example 13, the method of Examples 1 or 12 is optionally performed such that automatically delivering a response comprises adjusting cardiac function management of the subject.

In Example 14, the method of Example 1 optionally includes trending periodic breathing during activity indication, wherein the periodic breathing indication occurs two or more times within a specified duration and the duration comprising at least two days.

In Example 15, the method of Example 1 optionally includes generating an alert in response to a change in value, the change in value comprising an increase or decrease in the cycle length of the periodic breathing during activity indication.

In Example 16, the method of Example 1 optionally includes generating an alert in response to a change in value, the change in value comprising an increase or decrease in the amplitude of the periodic breathing during activity indication.

In Example 17, the method of Example 1 is optionally performed such that analyzing how well the lung ventilation data fits the model comprises updating the model using recent monitored respiration of the subject.

Example 18, describes an apparatus comprising an activity detector, a respiration monitor, and a processor circuit, coupled to at least one of the activity detector and the respiration monitor. The activity detector is configured to detect a physical activity indication of a subject. The respiration monitor is configured to obtain respiration pattern data of the subject during the activity. The processor is configured to analyze how well the respiration pattern data during activity fits a model to provide a resulting goodness of fit indication, the processor configured to use the goodness of fit indication to determine and provide a periodic breathing during activity indication.

In Example 19, the apparatus of Example 18 optionally includes an exertion module, operatively coupled to the activity detector. The exertion module includes a timer circuit and is configured to generate a sustained activity indication in response to physical activity. The respiration monitor is optionally configured to be enabled to obtain lung respiration pattern data during the sustained activity.

In Example 20, the apparatus of at least one of Examples 18-19 optionally configured with the period of sustained physical activity that comprises an exertion or duration specified by a user.

In Example 21, the apparatus of at least one of Examples 18-19 optionally includes a trending module, operatively coupled to the exertion module. The trending module is configured to trend periodic breathing indication occurring two or more times within a specified duration and the duration comprising at least two days.

In Example 22, the apparatus of Example 18 optionally includes an alert circuit, operatively coupled to the respiration monitor. The alert circuit is configured to generate an alert indication in response to a change in value of at least one of an increased cycle length of the periodic breathing during the activity indication or an increased amplitude of the periodic breathing during activity indication.

In Example 23, the apparatus of Example 18 is optionally configured with the respiration monitor that comprises a respiration rate detector circuit. The respiration rate detector circuit is configured to calculate one or more of a respiration rate, a minute ventilation and a tidal volume from the subject.

In Example 24, the apparatus of Example 18 is optionally configured with the processor that comprises the model of a respiration signal comprising one or more of a respiration signal over time or a respiration signal within the frequency domain.

In Example 25, the apparatus of Example 18 is optionally configured such that the processor is configured to calculate a goodness of fit of the respiration pattern data to the model by applying one or more of a least squares analysis or a power spectrum analysis.

In Example 26, the apparatus of Example 18 is optionally configured such that the processor is configured to report a magnitude or frequency in response to the goodness of fit indication meeting at least one criterion.

In Example 27, the apparatus of Example 18 is optionally configured such that the processor is configured to update the model using recent monitored respiration of the subject.

Example 28 describes a system comprising means for monitoring respiration of a subject, means for detecting physical activity of the subject, means for obtaining a respiration pattern of the subject during the activity, means for analyzing how well the respiration pattern during activity fits a model, the analyzing providing a goodness of fit indication, means for identifying a periodic breathing during activity indication from the goodness of fit indication, and means for providing the periodic breathing during activity indication to a user or automated process.

In Example 29, the system of Example 28 optionally includes means for computing the goodness of fit indication using at least one of a least squares analysis or a power spectrum analysis.

In Example 30, the system of Example 28 optionally includes means for reporting a respiration pattern magnitude or frequency in response to the goodness of fit indication.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a block diagram illustrating generally an example of a system including an implantable device, which is typically wirelessly communicatively coupled by a communication module to an external local interface, which, in turn is communicatively coupled to an external remote server, such as over a wired or wireless telecommunications or computer network.

FIG. 2 is a diagram illustrating generally an example of portions of a technique for monitoring disordered breathing.

FIG. 3 is a diagram illustrating generally an example of how the indicators of disordered breathing density or frequency during sleep, exercise, and rest can be used.

FIG. 4 is a diagram illustrating generally an example of how such indicators can be used to form a combined metric.

FIG. 5 is a diagram, similar to FIG. 2, but illustrating a technique in which a periodic breathing (PB) event is detected, instead of the detecting of a disordered breathing (DB) event in FIG. 2.

FIG. 6 is a block diagram of an example, similar to FIG. 1, in which the implantable cardiac function management device includes a detector for another CHF symptom, such as a pulmonary fluid accumulation detector.

FIG. 7 is a block diagram of another example of an implantable cardiac function management device that includes an apnea detector and an apnea classifier.

FIG. 8 is a block diagram of another example of an implantable cardiac function management device that includes an exertion measurement circuit.

FIG. 9 is a diagram illustrating an example of a technique in which a periodic breathing event occurs during exercise using a model.

FIG. 10 is a graph illustrating an example of a respiration pattern of a subject with model data overlaying the respiration pattern.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

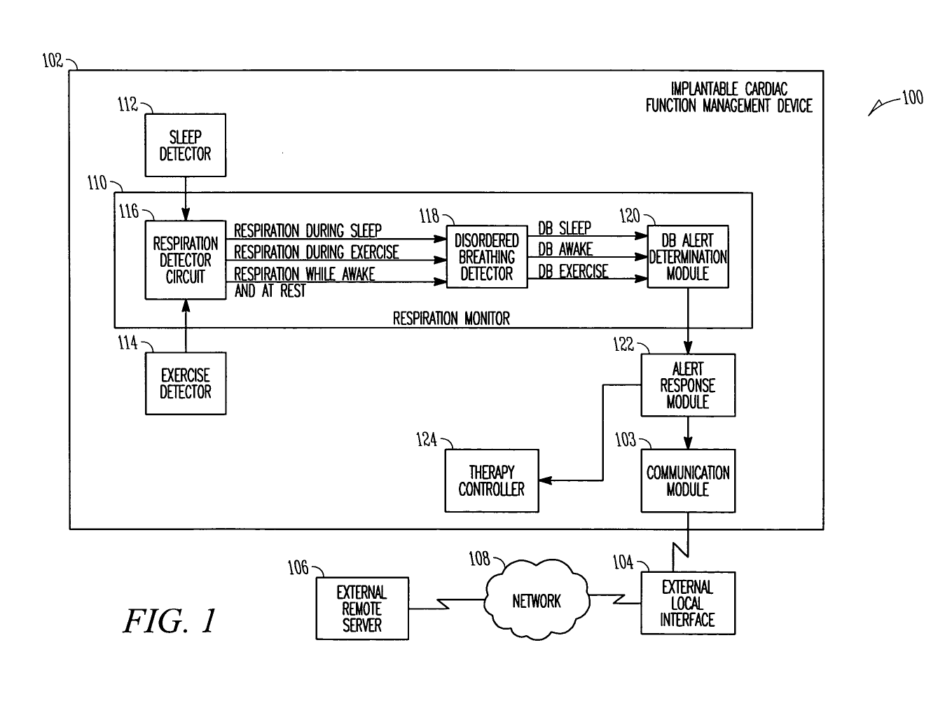

FIG. 1 is a block diagram illustrating generally an example of a system 100 including an implantable device 102, which is typically wirelessly communicatively coupled by a communication module 103 to an external local interface 104, which, in turn is communicatively coupled to an external remote server 106, such as over a wired or wireless telecommunications or computer network 108. In certain examples, the implantable device 102 includes an implantable cardiac function management device, such as a pacer, cardioverter, defibrillator, cardiac resynchronization therapy (CRT) device, or a combination device that combines these or other functions, such as patient monitoring, therapy control, or the like.

In this example, the implantable device 102 can include a hermetically sealed housing to carry electronics, and can include a respiration monitor 110, a sleep detector circuit 112, and an exercise detector circuit 114. In the example of FIG. 1, the respiration monitor 110 includes a respiration detector circuit 116 that transduces a subject's breathing into an electrical signal representative of such breathing. An example of a respiration detector circuit 116 is a transthoracic impedance sensor, which detects variations in transthoracic impedance as a subject inhales and exhales, such as described in Hartley et al. U.S. Pat. No. 6,076,015, which is incorporated herein by reference in its entirety, including its description of an impedance-based respiration detector. In certain examples, a respiration detector circuit 116 can provide lung ventilation data such as through the use of detecting thorax conductivity changes and rib cage movement or identification of the amount of lung volume during a given activity, known as tidal volume. In other examples, a respiration signal can be derived from detected heart sounds, detected blood pressures, or one or more other proxy parameters. An example of a sleep detector 112 is described in Yousafali Dalal et al.

U.S. patent application Ser. No. 11/458,602, entitled “SLEEP STATE DETECTION”, filed on Jul. 19, 2006 (Attorney Docket Number 279.B65US1), which is incorporated by reference in its entirety, including its description of a sleep detector. An example of an exercise detector 114, is an accelerometer, which can be configured to produce a signal representative of the subject's physical activity, which, in turn, can be signal-processed to obtain an indication of a representative level of activity. For example, a rate-responsive pacer may already include an accelerometer-based exercise detector to determine a patient activity level, so that the pacing rate can be adjusted according to the patient activity level to adjust cardiac output according to a perceived metabolic need for such cardiac output. In certain examples, physical activity exceeding an exertion level or being sustained for a duration specified by a user can be used to determine physical activity has occurred. For example, an exertion exceeding 20 mGs (1 mG=( 1/1000)*gravitational acceleration) for a duration exceeding three minutes may indicate physical activity. In other examples, a range of 20-30 mGs may indicate a walking subject.

In the example shown in FIG. 1, the respiration detector circuit 116 can receive a sleep or awake indication from the sleep detector 112, and an exercise or resting indication from the exercise detector 114, to detect physical activity. The respiration detector circuit 116 can output responsive signals indicative of respiration during sleep, respiration during exercise, and respiration while awake and at rest. In the example of FIG. 1, such signals are received by a disordered breathing detector 118. Although FIG. 1 has been illustrated, for conceptual clarity, as having separate signals representing respiration during sleep, respiration during exercise, and respiration while awake and at rest, it is understood that the disordered breathing detector 118 can alternatively be implemented to receive a single respiration signal, together with sleep/awake information from the sleep detector 112 and exercise/rest information from the exercise detector 114.

However implemented, the disordered breathing detector 118 will typically compute a separate indication of the amount of disordered breathing occurring during at least one of sleep, exercise, and resting awake states, which can be denoted as DBₛₗₑₑₚ, DB_{exercise}, and DBᵣₑₛₜ, respectively. More typically, the disordered breathing detector 118 will typically compute separate indications of the amount of disordered breathing occurring during at least two of sleep, exercise, and resting awake states, which can be denoted as DBₛₗₑₑₚ, DB_{exercise}, and DBᵣₑₛₜ, respectively. Even more typically, the disordered breathing detector 118 will compute three separate indications of the amount of disordered breathing occurring during each of sleep, exercise, and resting awake states.

Such disordered breathing can include incidences of apnea. Apnea occurs when breathing stops for a brief period, which may then be followed by hyperventilation. In certain examples, cessation of breathing for a period of at least 10 seconds is deemed an apnea event. Sleep disordered breathing can also include incidences of hypopnea. Hypopnea occurs when breathing amplitude decreases for a brief period, which may then also be followed by hyperventilation. In certain examples, a drop in breathing amplitude by at least 30%-50% (and which does not constitute apnea) for a period of at least 10 seconds is deemed a hypopnea event. An apnea-hypopnea index (AHI) can be defined as the number of apnea and hypopnea events during a period of sleep divided by the duration of that period of sleep.

However, disordered breathing can also include hypopnea events that can occur even if the patient is awake, such as when the patient is awake and resting, or when the patient is awake and exercising. Whether when awake or asleep, if such hypopnea events become frequent enough, they can be deemed periodic breathing, which can be conceptualized as a recurring cycle of a hypopnea event, which followed by a period of respiration (which is often hyperventilation to offset the hypopnea). Hypopnea events or periodic breathing occurring during exercise, for example, is believed to have different clinical significance than such incidences occurring during sleep, and such incidences occurring when the subject is awake but at rest. Periodic breathing during exercise is sometimes referred to as exertional oscillatory ventilation (EOV). In general, patients having AHI<30 and no EOV are believed to expect a better survival rate than patients with EOV alone, who are believed, in turn, to expect a better survival rate than patients with AHI>30 alone (but no EOV), who are believed, in turn, to expect a better survival rate than patients with combined breathing disorder (CBD), that is, both AHI>30 and EOV. Heart failure subjects experiencing increased duration or magnitude of the periodic breathing may indicate an increase in severity of their heart condition. Thus, by categorizing disordered breathing, such as according to sleep, exercise, and resting awake states, a more accurate patient wellness indicator can be created than by computing disordered breathing without distinguishing between whether such disordered breathing occurs during a sleep, an exercise, or a resting awake state. Such more specific wellness indicator(s) can be provided to an alert determination module 120 and used to provide a more accurate alert, such as to the patient, to the patient's physician, or to the patient's personal medical device that initiates or adjusts one or more responsive therapies. In the example of FIG. 1, the alert determination module 120 can provide resulting alert to an alert response module 122, which can sound a buzzer, or communicate an alert via communication module 103 to external local interface 104 (e.g., a patient interface), or to an external remote server 106, which can provide remote monitoring and notification of the patient or the patient's physician. Alternatively or additionally, in the example of FIG. 1, the alert response module 122 can provide closed-loop feedback to a therapy controller 124, which can initiate or adjust one or more congestive heart failure (CHF) or other therapies to be automatically delivered to the patient, such as cardiac resynchronization therapy (CRT), drug delivery, or any other suitable responsive therapy. Examples of CRT include, without limitation, adjusting AV delay, adjusting interventricular pacing delay, adjusting intraventricular pacing delay, adjusting intraventricular electrode selection, adjusting cardiac contractility modulation (CCM) therapy, or the like.

The disordered breathing detector 118 can be configured to count a number of apnea or hypopnea events, and to compute an overall unweighted disordered breathing severity indication. In certain examples, this disordered breathing severity indication can be determined using a “density” (e.g., frequency or rate of occurrence) of such events per unit time. Similarly, the disordered breathing detector 118 can be configured to compute separate disordered breathing severity indications for sleep, exercise, and awake and resting states. Such separate disordered breathing severity indications for sleep, exercise, and awake and resting states can be separately (e.g., differently) weighted and combined into an overall weighted disordered breathing severity indication, which can in certain examples represent a density of such events per unit time. The disordered breathing severity indication can additionally or alternatively use other information to determine severity, such as a duration of a disordered breathing episode, a measure of the amount of decrease of the respiration amplitude during the episode, or any other information that is indicative of the severity of the disordered breathing episode.

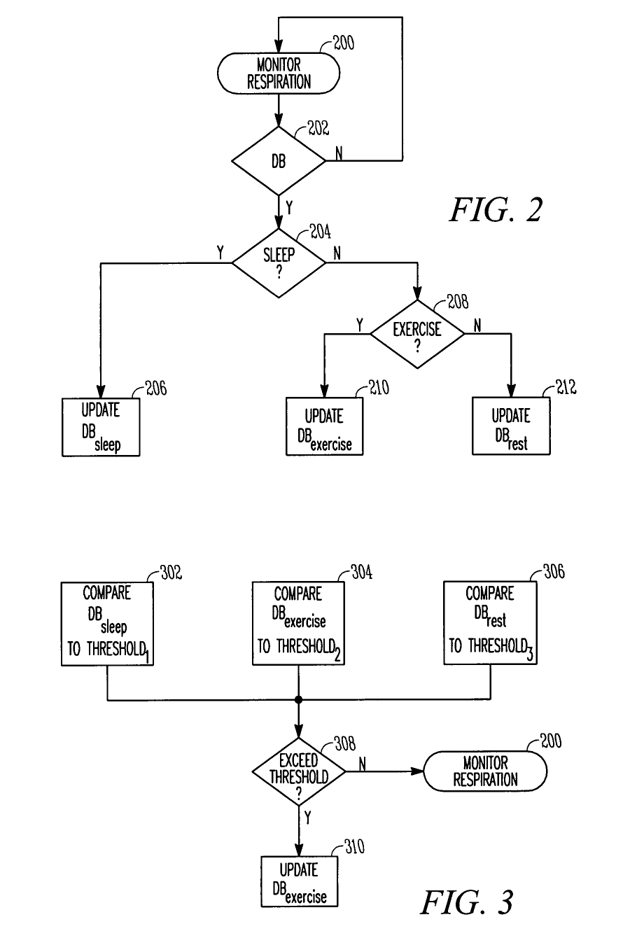

FIG. 2 is a diagram illustrating generally an example of portions of a technique for monitoring disordered breathing. In the example of FIG. 2, at 200, respiration is monitored for incidences of disordered breathing, such as an apnea event or a hypopnea event, as discussed above. At 202, if such a disordered breathing (DB) event is detected, then at 204, it is determined whether the subject was sleeping, otherwise process flow returns to 200. At 204, if the subject was sleeping when the DB event was detected, then a DBsleep density or severity indicator is updated at 206. In certain examples, this can involve computing an inverse of a time period since the last DB event was detected in either a sleep, exercise, or resting state, and including this value in a buffer of the N most recent similar values occurring during sleep. At 204, if the subject was not sleeping when the DB event was detected, then at 208 it is determined whether the subject was exercising when the DB event was detected. If so, then at 210, a DBexercise density or severity indicator is updated, similar to the updating of the DBsleep density or severity indicator at 206. Otherwise, then at 212, a DBrest density or severity indicator is updated, similar to the updating of the DBexercise density or severity indicator at 210 and the DBsleep density or severity indicator at 206. In this manner, separate indications of the severity or density over time of disordered breathing are computed for the sleep, exercise, and awake but resting states.

FIG. 3 is a diagram illustrating generally an example of how the DBsleep density or severity indicator, the DBexercise density or severity indicator, and the DBrest density or severity indicator can be used. At 302, the DBsleep density or severity indicator is compared to a threshold value, which can be programmed specifically for the DBsleep density or severity indicator. At 304, the DBexercise density or severity indicator is compared to a threshold value, which can be programmed specifically for the DBexercise density or severity indicator. At 306, the DBrest density or severity indicator is compared to a threshold value, which can be programmed specifically for the DBrest density or severity indicator. At 308, if at least two of these comparisons exceed their respective threshold value, then an alert is triggered at 310, otherwise process flow returns to 200, where respiration monitoring continues.

Variations on this technique are also possible. For example, at 308, the condition could be defined such that if at least one of the comparisons exceeds its respective threshold value, then an alert is triggered at 310. Alternatively, at 308, the condition could be defined such that all three comparisons must exceed their respective threshold values for the alert to be triggered at 310. In any of these various examples, the corresponding threshold can optionally be set using a long-term average or baseline of the particular one of the DBsleep density or severity indicator, the DBexercise density or severity indicator, and the DBrest density or severity indicator. In this manner, an alert will only be triggered if there is a more than insubstantial (e.g., 3 standard deviations above baseline) change in one or more than one of such density or severity indicators, depending on which test condition is used.

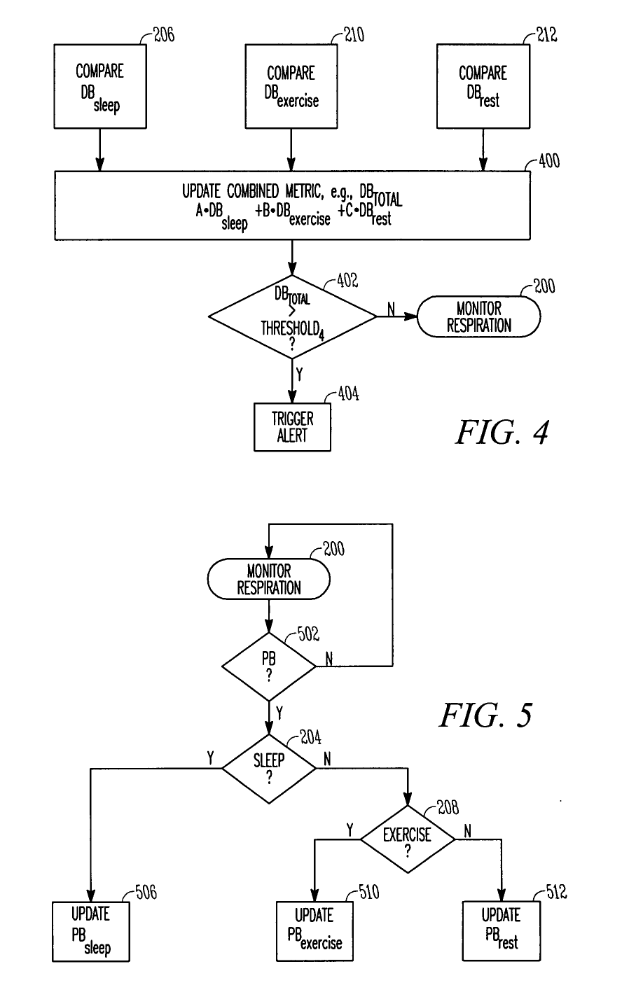

FIG. 4 is a diagram illustrating generally an example of how the updated DBsleep indicator, the DBexercise indicator, and the DBrest indicator can be used. After these respective indicators are updated, such as at 206, 210, and 212, respectively, then at 400, a combined metric DBtotal is updated, such as according to DBtotal=A·DBsleep+B·DBexercise+C·DBrest, where A, B, C are independently specified scaling values. Then, at 402, the combined metric DBtotal is compared to a corresponding threshold value. If, at 402, DBtotal exceeds its corresponding threshold value, then at 404 an alert is triggered, otherwise process flow returns to the respiration monitoring at 200.

In certain variations of the above technique, the combined metric DBtotal is logged, such as on a daily basis. Moreover, the threshold to which the DBtotal metric is compared can be set based on a baseline long-term value of the same metric, or based on the baseline value and variance (e.g., threshold at +3 standard deviations above baseline).

FIG. 5 is a diagram, similar to FIG. 2, but illustrating a technique in which a periodic breathing (PB) event is detected at 502, instead of detecting a disordered breathing (DB) event at 202 of FIG. 2. A PB event can be conceptualized as a DB event (e.g., apnea or hypopnea) that is recurring often enough and with sufficient periodicity to be considered periodic breathing instead of a series of isolated DB events. One example of disordered breathing is described in Yachuan Pu et al. U.S. patent application Ser. No. 11/392,365 entitled “PERIODIC DISORDERED BREATHING DETECTION”, filed on Mar. 28, 2006 (Attorney Docket No. GUID.242.A1), which is incorporated herein by reference in its entirety, including its description of detecting periodic breathing. In brief, periodic breathing can be detected by rectifying the respiration signal, and lowpass filtering the rectified signal (e.g., such as with a moving average) to obtain an “envelope” signal. The resulting envelope signal can be further filtered (e.g., highpass filtered to remove baseline wander) and then tested for amplitude variations of sufficient magnitude to constitute periodic breathing. Periodic breathing density or severity indicators can be computed for sleep, exercise, or resting states at 506, 510, and 512 respectively, similarly to the above description of computing disordered breathing density or severity indicators for similar states at 206, 210, and 212, respectively of FIG. 2.

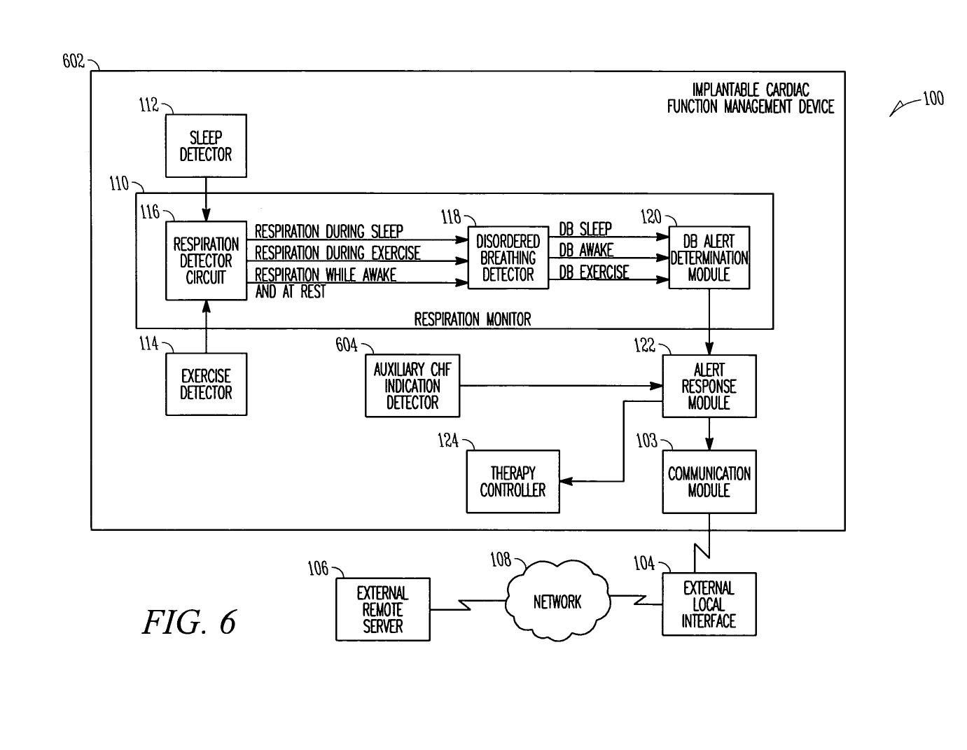

Disordered breathing and periodic breathing can be symptomatic of congestive heart failure (CHF). Therefore, in generating any alert based on disordered breathing or periodic breathing, it may be desirable to qualify or otherwise base such alert on one or more other detected symptoms of CHF. For example, FIG. 6 illustrates a block diagram of an example, similar to FIG. 1, in which the implantable cardiac function management device 602 includes an auxiliary CHF indication detector 604. As an illustrative example, the auxiliary CHF indication detector 604 includes a pulmonary fluid accumulation detector to detect accumulation of pulmonary fluid, which is another symptom of CHF. The pulmonary fluid accumulation detector can measure transthoracic impedance, which will tend to decrease as pulmonary fluid accumulates in the thorax. The pulmonary fluid accumulation detector can itself include a posture detector, to reduce or eliminate the effect of postural changes in thoracic impedance measurements to get a more accurate representation of pulmonary fluid accumulation. Other examples of the auxiliary CHF indication detector 604 include a pulmonary artery pressure sensor, a heart sound sensor, a heart rate variability (HRV) sensor, a patient weight indicator (which may receive information communicated from an external weight scale), a patient activity sensor, or the like. For example, increased pulmonary artery pressure can indicate an onset of pulmonary embolism, or blood clotting, as a sign of increased CHF conditions. In another example, heart rate variability sensor can indicate diastolic or systolic dysfunction, or both, as a sign of increased CHF conditions. The auxiliary CHF indication detector 604 can also combine multiple such sensors to provide various indications of CHF.

In the example in which the auxiliary CHF indication detector 604 includes a pulmonary fluid accumulation detector, an indication of detected pulmonary fluid can be provided to the alert response module 122. The indication of detected pulmonary fluid can be used to generate a separate alert, or to qualify an alert based on disordered or periodic breathing, such that both pulmonary fluid accumulation and one or both of disordered or periodic breathing is required in order to trigger the responsive alert. Alternatively or additionally, the pulmonary fluid accumulation indication (or any other appropriately weighted indications of one or more other CHF symptoms) can be appropriately weighted and combined with the disordered breathing indication (or any other appropriately weighted indications of one or more other CHF symptoms) to create a CHF status indicator representative of a CHF patient's wellness or sickness based on multiple symptoms.

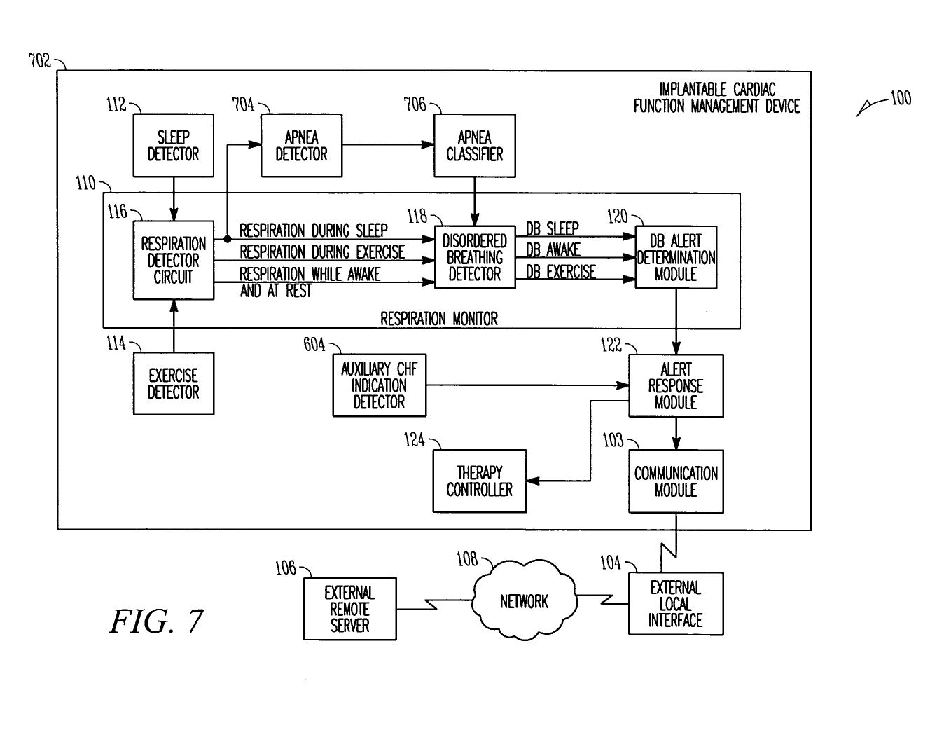

FIG. 7 is a block diagram of another example of an implantable cardiac function management device 702 that includes an apnea detector 704 and an apnea classifier 706. In this example, the apnea detector 704 receives respiration during sleep information from the respiration detector 116, and detects incidences of apnea. The apnea detector 704 provides information about detected incidences of apnea to the apnea classifier 706, which classifies the apnea, for example, as obstructive sleep apnea (OSA) or central sleep apnea (CSA). One illustrative example of a sleep apnea detector and classifier is described in Patangay et al. U.S. patent application Ser. No. 11/425,820, filed on Jun. 22, 2006, entitled APNEA TYPE DETERMINING APPARATUS AND METHOD (Attorney Docket No. 279.C24US1), which is incorporated herein by reference in its entirety, including its description of an apnea detector and classifier.

Since CSA is more likely than OSA to be indicative of CHF, the apnea classification information provided by the apnea classifier 706 to the disordered breathing detector 118 can be used to either: (1) qualify the disordered breathing during sleep density or severity indicator, such that only CSA episodes are counted, and CSA episodes are not counted; or (2) provide separate disordered breathing during sleep density or severity indicators to separately count incidences of OSA and CSA, with the DB alert determination module 120 formulating its alert based on these separate indicators similar to the manner described above.

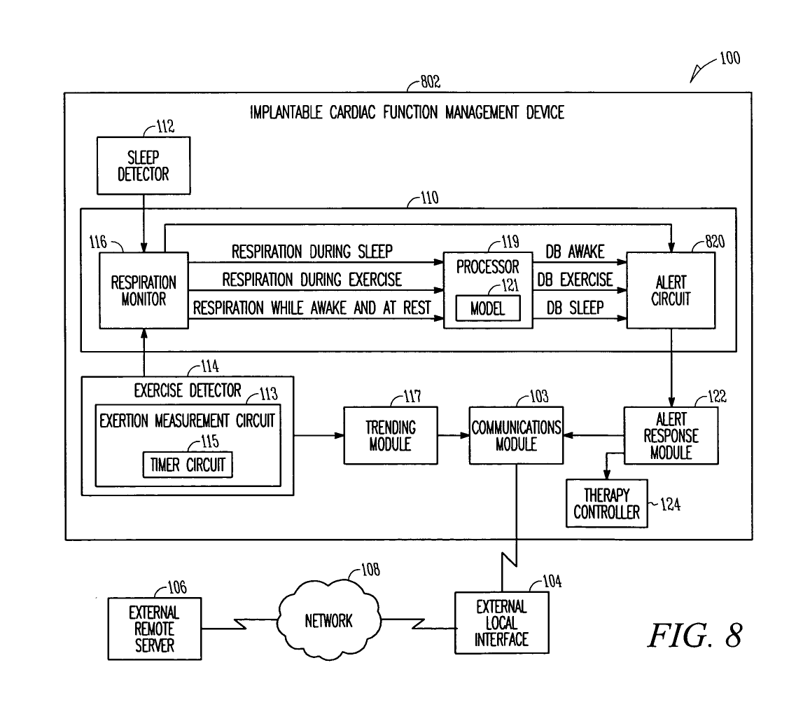

FIG. 8 is a block diagram of another example of an implantable cardiac function management device 802 that includes a sleep detector 112 and an exercise detector 114. In this example, the exercise detector 114 includes an exertion measurement circuit 113 having a timer circuit 115. The exertion measurement circuit 113 measures an amount and duration of exertion of detected physical activity as timed by the timer circuit 115. Exertion measurement can be made in units of mG, or millionths of gravitational force. For example, the exercise detector 114 may detect physical activity by the subject but either the exertion level is very low (e.g., below 20 mGs) or the activity lasts for a very short time (e.g., 2 minutes or less). Under such circumstances, an exercise state is not declared, in certain examples. Accordingly, the respiration measured by the respiration monitor 116 during such time is not considered as having occurred during an exercise state.

The exertion measurement circuit 113 can also provide exertion or duration or other timing information to the trending module 117 so that this information can be trended over time, such as for display or storage. Trending data can include, for example, trending magnitude or frequency over time. Monitoring a trend of respiration magnitude or frequency over time can indicate a change in the patient's condition over time or can even characterize a respiration related event, such as when the trend is compared to at least one specified criterion. Information about respiration during exercise can be transmitted from the respiration detector 116 to the processor 119. The processor 119 can calculate lung respiration data such as tidal volume or breathing rate and can include a time or frequency domain disordered breathing model 121 representing one or more instances of disordered breathing. When, during exercise, breathing signal information matches or resembles the disordered breathing model 121, the alert circuit 820 can output a resulting disordered breathing alert signal to the alert response module 122. In certain examples, the alert response module 122 is configured to communicate this alert internally or externally, such as by using the therapy controller 124 or communications module 103. In certain examples, the processor 119 can compare, such as during sleep or awake rest states, a respiration pattern to the disordered breathing model 121. In certain examples, a separate model exists for each state (exercise, sleep or awake rest) and the particular model is identified by the exercise and sleep detector or if the model 121 updates upon a change in states. When determining whether respiration matches the model 121, a goodness of fit determination can be made, such as by using one or more calculations, such as a least squares or power spectrum analysis. The goodness of fit determination can be made between the model and one or more aspects of the respiration pattern, such as respiration magnitude, respiration rate, respiration cycle length, minute ventilation, or the like.

One approach to establishing disordered breathing during exercise is to establish an exertion threshold and monitor the number of times within a specified duration that the threshold is exceeded or over what duration the threshold has continuously been exceeded. In contrast, in certain examples of the present approach, the model 121 can be established when the exercise detector 114 has identified that sustained activity has occurred, according to specified criteria of exertion and duration. The respiration data can be collected and used by the processor 119 to calculate a model 121 comprising start parameter values derived from measured respiration data over time. An example of a model least squares fitting formula, formula (1), is as follows:
Y=A*sin(ωt−φ) (1)

Where (A) represents the magnitude of oscillation of one or more aspects of the respiration signal, (ω) is the frequency of oscillation, (t) represents the duration in time, and (φ) represents the phase lag term. Once the model 121 is established, later respiration patterns can be compared to the model 121. In certain examples, disordered breathing can be identified using the model 121. Using a goodness of fit calculation, such as Chi-Square, it can be determined whether a new respiration pattern exhibits similarity to a disordered breathing pattern. Chi-Square can identify a discrepancy between observed values and the values expected under a given model. One such Chi-Square formula, formula (2), is:

$${X}^{2} = \sum \frac{{( O - E )}^{2}}{E} \qquad ( 2 )$$

Where (O) represents an observed frequency, such as respiration data, and (E) represents an expected frequency, such as the model 121 frequency. The resulting value of X can then be compared to a Chi-Square distribution table to determine the goodness of fit. A value of X² that is close to zero represents a high probability of goodness of fit. In certain examples, the goodness of fit can be used to identify instances of periodic or other disordered breathing. A disordered breathing indication can be generated to produce a trend, an alert, or control applying therapy. The model 121 can also be updated or adjusted over time such as to maintain accuracy or when the goodness of fit does not indicate disordered breathing in the respiration data for long periods of time.

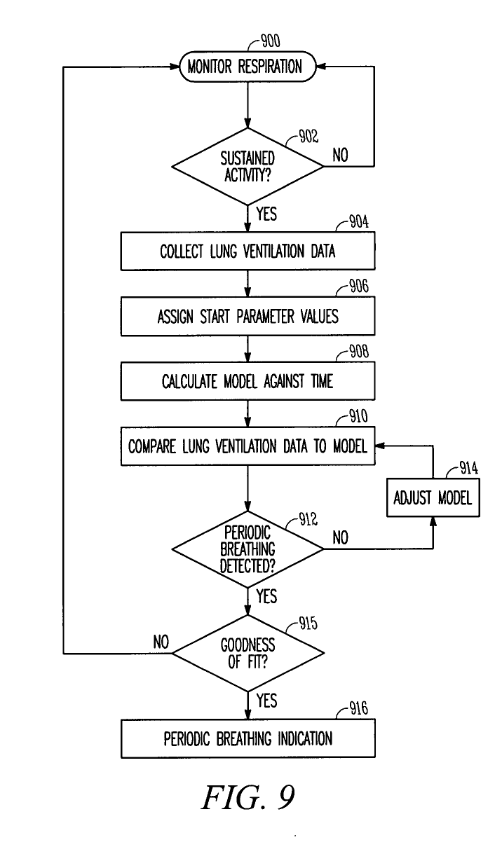

FIG. 9 is a diagram illustrating generally an example of portions of a technique for monitoring disordered breathing during exercise. In the example of FIG. 9, at 900, respiration is monitored for incidences of disordered breathing. At 902, if sustained physical activity is detected (e.g., exercise), then at 904, lung ventilation data is collected. Otherwise, monitoring is continued at 900. At 906, the lung respiration data collected at 904 can be assigned starting parameter values to be used by the processor, at 908 to calculate the model against time. At 910, the lung ventilation data collected at 904 can be compared to the model. At 912, if periodic breathing is detected using the model, then, at 915, if the detected periodic breathing has a statistically significant fit to the model, then at 916, a periodic breathing indication can be generated. Otherwise, at 914, the model can be adjusted (such as to accommodate a changed patient condition, e.g., period length of the detected disordered breathing, or magnitude of breathing patterns showing shallow or deep breathing during respiration monitoring). Otherwise, at 915, if the detected periodic breathing does not have a statistically significant fit to the model, monitoring is continued at 900. For example, if a noisy signal is present, the periodic breathing detection may identify some periodicity which has no physiologic correlation to the lung ventilation data. The statistical fit determination permits such false indications to be ignored. Without being bound by theory, it is believed that instances of increased respiration cycle length or increased respiration amplitude during exercise, for patients diagnosed with congestive heart failure (CHF), can represent increased degree of severity of CHF. In this manner, instances of disordered breathing during exercise can indicate early signs of deteriorated patient conditions while updating the test model.

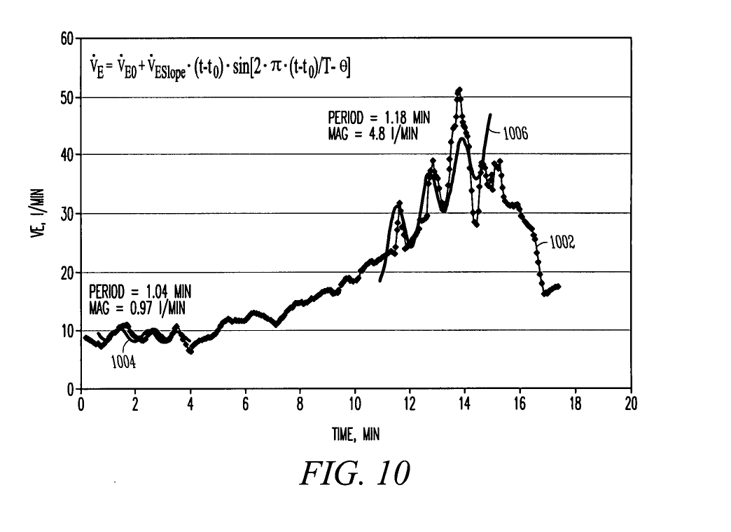

FIG. 10 is a graphical display illustrating generally an example of a model 121 of respiration data having components of both magnitude and frequency in which least squares fitting, formula (1), is used and the data points are plotted over time. The x-axis represents duration and the y-axis represents exertion. Respiration data plot 1002 represents the respiration data of a patient under both awake rest and exercise conditions. Awake rest model plot 1004 is shown overlying the respiration data plot 1002 during the first four minutes and indicates a goodness of fit period=1.04 and magnitude=0.97. Exercise model plot 1006 is shown overlying the respiration data plot 1002 between eleven to fifteen minutes and indicates a goodness of fit period=1.18 and magnitude=4.8. Disordered breathing components can be further represented in the model 121, thereby providing an indication of disordered breathing episodes such as periodic breathing.

Although the above description has emphasized an example in which processing is generally carried out within an implantable device, information derived from the respiration signal obtained from the implantable device can be communicated to external local interface 104 or external remote server 106 to perform such processing at such other locations. Moreover, such processing can include information from one or more devices that are not implanted. For example, a body weight measurement as measured by an external weight scale could be combined with a disordered breathing indication obtained from an implantable cardiac function management device, e.g., during processing at external remote server 106, to generate a CHF wellness indicator or to trigger an alert or responsive therapy.

In certain examples, information from the disordered breathing detector 118 (e.g., indications of disordered breathing density or severity in sleep, exercise, or awake but resting states) can be provided to the communication module 103, and communicated to the external local interface 104 or the external remote server 106, such as for storage or for display on a monitor, for example, as separate trends of disordered or periodic breathing density or severity in sleep, exercise, or awake but resting states, or as histograms of disordered or periodic breathing density or severity in sleep, exercise, or awake but resting states, or in any other useful form.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising ” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. A method comprising:
monitoring respiration of a subject;
detecting physical activity of the subject;
obtaining a respiration pattern of the subject during the activity;
analyzing how well the respiration pattern fits a model, the analyzing providing a goodness of fit indication;
identifying a disordered breathing during activity indication from the goodness of fit indication; and
providing the disordered breathing during activity indication to a user or automated process.

2. The method of claim 1, wherein the obtaining a respiration pattern comprises determining tidal volume of the subject.

3. The method of claim 1, wherein the obtaining a respiration pattern comprises determining respiration rate of the subject.

4. The method of claim 1, wherein detecting physical activity comprises detecting a period of sustained physical activity exceeding an exertion or duration specified by a user.

5. The method of claim 4, wherein the specified exertion comprises activity exceeding at least 20 mGs.

6. The method of claim 4, wherein the specified duration comprises at least three minutes.

7. The method of claim 1, wherein analyzing how well the respiration pattern fits the model comprises using a model of a respiration signal over time.

8. The method of claim 1, wherein analyzing how well the respiration pattern fits the model comprises using a model of a respiration within the frequency domain.

9. The method of claim 1, wherein providing the goodness of fit indication comprises applying one or more of a least squares analysis or a power spectrum analysis to obtain the goodness of fit indication.

10. The method of claim 1, comprising reporting a respiration pattern magnitude, a respiration pattern cycle rate, or a respiration pattern cycle length in response to the goodness of fit indication meeting at least one criterion.

11. The method of claim 1, comprising automatically delivering a response to the subject in response to the periodic breathing during activity indication.

12. The method of claim 1, comprising trending periodic breathing during activity indication, wherein the periodic breathing indication occurs two or more times within a specified duration and the duration comprising at least two days.

13. The method of claim 1, comprising generating an alert in response to a change in value, the change in value comprising an increase or decrease in the cycle length of the periodic breathing during activity indication.

14. The method of claim 1, comprising generating an alert in response to a change in value, the change in value comprising an increase or decrease in the amplitude of the periodic breathing during activity indication.

15. The method of claim 1, wherein analyzing how well the lung ventilation data fits the model comprises updating the model using recent monitored respiration of the subject.

16. A system comprising:
an activity detector, configured to detect a physical activity indication of a subject;
a respiration monitor, configured to obtain respiration pattern data of the subject during the activity;
a processor circuit, coupled to at least one of the activity detector and the respiration monitor, the processor configured to analyze how well the respiration pattern data during activity fits a model to provide a resulting goodness of fit indication, the processor configured to use the goodness of fit indication to determine and provide a periodic breathing during activity indication.

17. The system of claim 16, comprising an exertion module, operatively coupled to the activity detector, the exertion module including a timer circuit and configured to generate a sustained activity indication in response to physical activity, and wherein the respiration monitor is configured to be enabled to obtain lung respiration pattern data during the sustained activity.

18. The system of claim 17, wherein the period of sustained physical activity comprises an exertion or duration specified by a user.

19. The system of claim 17, comprising trending module, operatively coupled to the exertion module and configured to trend periodic breathing indication occurring two or more times within a specified duration and the duration comprising at least two days.

20. The system of claim 16, comprising an alert circuit, operatively coupled to the respiration monitor, the alert circuit configured to generate an alert indication in response to a change in value of at least one of an increased cycle length of the periodic breathing during activity indication or an increased amplitude of the periodic breathing during activity indication.

21. The system of claim 16, wherein the respiration monitor comprises a respiration rate detector circuit, configured to calculate one or more of a respiration rate, a minute ventilation or a tidal volume from the subject.

22. The system of claim 16, wherein the processor comprises the model of a respiration signal comprising one or more of a respiration signal over time or a respiration signal within the frequency domain.

23. The system of claim 16, wherein the processor is configured to calculate a goodness of fit of the respiration pattern data to the model by applying one or more of a least squares analysis or a power spectrum analysis.

24. The system of claim 16, wherein the processor is configured to report a magnitude or frequency in response to the goodness of fit indication meeting at least one criterion.

25. The method of claim 16, wherein the processor is configured to update the model using recent monitored respiration of the subject.
